(19) United States
(12) Patent Application Publication
(10) Pub. No.: US 2026/0183328 A1
(43) Pub. Date: Jul. 2, 2026

(54) COMPOSITIONS AND METHODS FOR TREATING CANCER

(71) Applicant: Celularity Inc., Florham Park, NJ (US)

(72) Inventors: Marina GERGUES, Florham Park, NJ (US); Alexandra HARIRI, Florham Park, NJ (US); Qian YE, Florham Park, NJ (US); Yuechao ZHAO, Florham Park, NJ (US); Lin KANG, Florham Park, NJ (US); Shuyang HE, Florham Park, NJ (US); Robert J HARIRI, Florham Park, NJ (US)

(73) Assignee: Celularity Inc., Florham Park, NJ (US)

(21) Appl. No.: 19/129,085

(22) PCT Filed: Nov. 15, 2023
(86) PCT No.: PCT/US2023/079857

§ 371 Date: May 12, 2025

Related U.S. Application Data
(60) Provisional application No. 63/383,759, filed on Nov. 15, 2022.

(51) Int. Cl. A61K 31/7105; A61K 9/50; A61K 45/06; A61P 35/00; C12N 15/113

(52) U.S. Cl. CPC A61K 31/7105; A61K 9/5068; A61K 45/06; A61P 35/00; C12N 15/1136; C12N 2310/14

(57) ABSTRACT

Provided herein are novel exosomal compositions that exhibit cytotoxicity and method of treating cancer that utilize such exosomal compositions.

### RELATED APPLICATIONS

This application claims priority of U.S. Provisional Patent Application Ser. No. 63/383,759 filed 14 Nov. 2022, the entire content of which is hereby incorporated herein by reference.

### BACKGROUND

Elevated TGF-β levels have been implicated in inhibiting the penetration of T-cells into the regions in proximity to tumors (Liu et al., Cancer Cell Int (2015) 15:106-112 “Cyclooxygenase-2 promotes tumor growth and suppresses tumor immunity” ; Zelenay et al. Cell (2015) 162:1257-1270 “Cyclooxygenase-Dependent Tumor Growth through Evasion of Immunity”; and Mariathasan et al., Nature (2018) 554; 544-548 “TGF attenuates tumour response to PD-L1 blockade by contributing to exclusion of T cells”; and 1-3).

In glioblastoma, TGF-β product and activity may increase, and potentially result in differences in responses to anti-TGF-β therapies in distinct subgroups of glioblastoma patients. TGF-β can enhance tumor growth, invasion, angiogenesis, and immunosuppression. Downregulation of TGF-β may inhibit cytokine-induced signaling pathways and transcriptional responses in transiently transfected human glioblastoma cells. Tumorigenicity of glioblastoma cells could also be reduced by RNAi-mediated TGF-β gene silencing. TGF-β can also inhibit antitumor immune responses by blocking maturation and function of professional APC and the synthesis of cytotoxic molecules such as perforin, granzymes, IFN-γ, and FasL in activated CTL. TGF-β also plays a role in tumor tolerance by recruiting Treg cells toward the primary tumor site as a means of immune evasion.134 High biological activity of the TGF-β-Smad pathway may contribute to the malignant phenotype of glioblastoma and confers poor prognosis to the patients. Glioblastoma Multiforme (GBM) is the most aggressive brain malignancy in adults, where the 5-year survival rate is less than 10%. Glioblastoma-initiating cells (GICs) are shown responsible for the initiation and recurrence of tumors.135 TGF-β induces the self-renewal capacity of GICs, but not of normal human neuroprogenitors, through the Smad and subsequent activation of the Janus Kinase-Signal Transducer and Activator of Transcription (JAK-STAT) pathways. Study also suggests that TGF-β downregulates NKG2D in the sera of glioblastoma patients, an important receptor involved in specific killing of transformed cells by CTLs, suggesting that blocking TGF-β may have further therapeutic benefit.136

Efforts have been made to deliver therapeutic agents that down-regulate or inhibit expression of TGF-β in tumor cells in order to suppress such cells'tumor immunity such that such cells are more amenable to destruction via a cell mediated response. However, localized drug delivery and specific targeting are two major problems researchers and clinicians must still confront. Indeed, one of the most challenging issues in anti-cancer and anti-inflammatory therapy continues to be achieving delivery of therapeutic agents to specific inflammatory cells and tumor cells in vivo and then, upon delivery, having the particular therapeutic agent retain its activity. For example, in spite of the development of therapeutic agents that preferentially target inflammatory and cancer cells without harming normal tissues, the delivery of these agents to the brain continues to be a major challenge because of difficulty in penetrating the blood-brain barrier. Indeed, the further development of many therapeutic agents has been abandoned because sufficient therapeutic agent levels in the brain could not be achieved via the systemic circulation. Intranasal delivery has previously provided a noninvasive method for delivering therapeutic agents to the brain in some instances, but the quantities of therapeutic agents that are able to be administered via that route and that are transported directly from nose-to-brain continue to be very low. As a result, cancers such as GBM, for which therapeutic agents must cross the blood-brain barrier, have been very difficult to develop. Some efforts have been made to confront these problems via the further development of nanoparticle-therapeutic agent delivery systems. However, such efforts have had only limited success.

A particular example of a cell therapy for treatment cancer is the administration of Natural killer (NK) cells. These cells are cytotoxic lymphocytes that constitute a major component of the innate immune system. They are activated in response to interferons or macrophage-derived cytokines. The cytotoxic activity of NK cells is largely regulated by two types of surface receptors, which may be considered “activating receptors” or “inhibitory receptors,” although some receptors, e.g., CD94 and 2B4 (CD244), can work either way depending on ligand interactions. Cancer cells with altered or reduced level of self-class IMHC expression result in induction of NK cell sensitivity. Activated and expanded NK cells, and in some cases LAK cells, from peripheral blood have been used in both ex vivo therapy and in vivo treatment of patients having advanced cancer.

Natural killer (NK) cells may be more suitable as therapeutic effectors against highly heterogeneous solid tumors such as glioblastoma (GBM), because unlike T and B lymphocytes, they do not possess rearranged V(D)J receptors and are not restricted by MHC-bound antigen presentation, which is downregulated in many solid tumors. Instead, their effector function is dictated by the integration of signals received through germline-encoded receptors that can recognize multiple ligands on cancer targets without particular antigen specificity or requirement for co-stimulation. However, NK cells become irreversibly, immunologically unresponsive owing to tumor elaborated TGF-β as well as other immunosuppressive molecules.

NK cells are a unique class of immune cells, innately capable of targeting cancer cells and interacting with adaptive immunity. Celularity (Basking Ridge, NJ, USA) is currently developing from placental hematopoietic stem cells a cryopreserved, allogeneic, off-the-shelf, natural killer (NK) cell (CYNK-001) therapy as a potential treatment option for various hematologic cancers, solid tumors, and infectious disease. CYNK-001 cells demonstrate a range of biological activities expected of NK cells, including expression of perforin and granzyme B, cytolytic activity against hematological tumors and solid tumor cell lines, and secretion of immunomodulatory cytokines such as IFN-γ in the presence of tumor cells. CYNK-001 cells express NKG2D and CD94, as well as NK activating receptors DNAM1, NKp30, NKp46, and NKp44. CYNK-001 cells are disclosed and described in US published Patent application US20220265712, which is hereby incorporated by reference herein in its entirety. Naturally, the cytotoxicity of CYNK-001 cells could be enhanced if the tumor cell's ability to express TGF-β could be down-regulated

### SUMMARY OF THE DISCLOSURE

The instant disclosure is based upon the discoveries that, surprisingly and unexpectedly:
A. an exosomal composition comprising exosomes that:
   (a) have been secreted from placental derived natural killer (CYNK) cells; and
   (b) have been modified to comprise an siRNA nucleotide sequence targeting the expression of TGF-β in cancer cells;
has a cytotoxic activity; and
B. administration of an effective amount of:
   (i) the exosomal composition of A, and
   (a) a cell population of the placental derived natural killer (CYNK) cells from which the exosomes of the exosomal composition of A were secreted,
exhibit a synergistic cytotoxic effect compared with the cytotoxic effect observed upon the sole administration of the exosomal composition of A or the cytotoxic effect observed upon the sole administration of the placental derived natural killer (CYNK) cells.

Thus, the instant disclosure extends to an exosomal composition comprising exosomes that comprise the nucleotide sequence of GGCUCAAGUUAAAAGUGGATT (SEQ ID NO: 1), wherein the exosomes are secreted from placental derived natural killer (CYNK) cells. SEQ ID NO: 1 is an siRNA nucleotide sequence targeting the expression of TGF-β. In a particular embodiment, SEQ ID NO: 1 is contained within the exosomes. In a particular embodiment, the placental derived natural killer cell type having applications herein is CYNK-001, an allogeneic off the shelf cell therapy enriched for CD56+/CD3-NK cells expanded from human placental CD34+ cells. CYNK-001 is manufactured in a cryopreserved formulation that is thawed and diluted at the clinical site prior to dose preparation and direct infusion. CYNK-001 is packaged at 30 ×10⁶ cells/mL in a total volume of 20 mL cryopreservation solution containing 10% (w/v) human serum albumin (HSA), 5.5% (w/v) Dextran 40, 0.21% sodium chloride (NaCl) (w/v), 32% (v/v) Plasma-Lyte A, and 5% (v/v) dimethyl sulfoxide (DMSO). It is filled into the container closure, frozen using a controlled rate freezer, and cryopreserved.

The instant disclosure further extends to a method of down-regulating expression of TGF-β in cancer cells in a subject, comprising administering an effective amount of an exosomal composition of the instant disclosure.

In addition, the instant disclosure extends to a method for treating cancer, comprising administering to a subject in need thereof an effective amount an exosomal composition of the present disclosure and an effective amount of a population of placental CD34+ derived natural killer (CYNK) cells that secreted exosomes used in an exosomal composition of the instant disclosure. A variety of cancer types can be treated with a method of the instant Disclosure. Examples of such cancers include, but certainly is not limited to skin cancer, gastric cancer, head and neck cancer, colon cancer, breast cancer, brain cancer, lung cancer, as well as any combination of such cancers. In a particular embodiment, the cancer is a brain cancer, e.g., glioblastoma (GBM). Administration of an effective amount of an exosomal composition of the instant disclosure and an effective amount of a population of placental derived natural killer (CYNK cells can occur simultaneously, concomitantly, or temporally separate from each other, e.g., 1, 2, 3, 4, 5, 6, 7, 8, 9, 10, 11, 12, or more hours apart. In a particular embodiment, when administered concomitantly, or temporally separate from each other, an exosomal composition of the instant disclosure is administered first, and then a population of placental derived natural killer (CYNK) cells is administered.

Optionally, an effective amount of a chemotherapeutic agent can also be administered to the subject. There are numerous types and examples of chemotherapeutic agents having applications herein. Such a chemotherapeutic agent can be a small molecule, e.g., an alkylating antineoplastic agent, a topoisomerase inhibitor, a taxane, a hormone analog, an antimetabolite, a plant alkaloid, an epipodophyllotoxin, an anti-tumor antibiotic, an anthracycline, or any combination thereof. Similarly, a chemotherapeutic agent having applications herein can be a biologic, e.g., an antibody such as PD-1 antibody, a hormone, or a cell, such as a T-cell genetically modified to express a chimeric antigen receptor for a particular type of cancer (e.g., a “CAR T-Cell”), as well as any combination of these chemotherapeutic agents. The administration of the chemotherapeutic agent can occur concomitantly with the administration of an exosomal composition or a population of CYNK cells as described herein.

Moreover, the instant disclosure extends to a method of treating cancer, comprising administering a therapeutically effective amount of an exosomal composition of the instant disclosure.

These and other aspects of the present disclosure will be better appreciated by reference to the following drawings and Detailed Description.

### BRIEF DESCRIPTION OF THE DRAWINGS

FIG. 1. Flow Cytometric Analysis of CYNK-Exo. Top Figure: Exemplary dot plot of CYNK-Exo bound to beads, stained with FITC and phenotyped for surface markers such as CD81. Bottom table: Expression of CD81, HLA-I, HLA-II, CD11a, CD47, CD56, CD226 and FasL on CYNK-Exo.

FIG. 2. Assessing Isolation Methods of CYNK-Exo Based on Concentration and Size. (A) Protein concentration as determined by microBCA of three CYNK-Exo donors isolated by four different methods. (B) Concentration of particles per mL as determined by ZetaView of three CYNK-Exo donors isolated by four different methods. (C) Median size of particles as determined by ZetaView of three CYNK-Exo donors isolated by four different methods.

FIG. 3. Assessing Isolation Methods of CYNK-Exo Based on Size Distribution. Exemplary size distribution plots for one CYNK-Exo donor isolated by four different methods.

FIG. 4. Assessing Isolation Methods of CYNK-Exo Based on Cytokine Profile. Concentration of (A) granzyme A, (B) granzyme B and (C) perforin from conditioned media provided by PD or CYNK-Exo after different isolation methods.

FIG. 5. Cytotoxicity of CYNK-Exo on Cancer Cells. Cytotoxicity of CYNK-Exo against tumor cells using xCelligence RTCA at indicated doses and time points for A) U251, (B) LN-229, (C) NCI-N87 and (D) OE-19.

FIG. 6. LDH Release of Cancer Cells Upon CYNK-Exo Treatment. Cytotoxicity of CYNK-Exo against tumor cells based on LDH release at indicated doses and time points for (A) U251, (B) LN-229, (C) NCI-N87 and (D) OE-19.

FIG. 7. Annexin V and 7AAD Staining of Cancer Cells Upon CYNK-Exo Treatment. Percent live, apoptotic and dead cells as determined by annexin V and 7AAD staining in (A) U251, (B) LN-229, (C) NCI-N87 and (D) OE-19 after 24 hours of CYNK-Exo treatment.

FIG. 8. Loading CYNK-Exo with Tagged-siRNA. Flow cytometric analysis of (top) pseudo-transfected and (bottom) transfected siCYNK-Exo to indicate efficient uptake of tagged-siRNA into CYNK-Exo. (Left) The total bead population is initially gated, (middle) followed by beads bound to CYNK-Exo as indicated by FITC+ (right) and finally CYNK-Exo that are positive for Cy3-tagged siRNA, which are positive in the PE channel.

FIG. 9. Viability and Uptake of siCYNK-Exo by U251. (Left) Viability of U251 after direct siRNA transfection with negative control or TGF-β siRNA, or with treatment of negative or TGF-β siCYNK-Exo. (Right) Percent cells positive for tagged-siRNA after direct transfection with negative control or TGF-β siRNA or with treatment of negative or TGF-β siCYNK-Exo.

FIG. 10. Viability and Uptake of siCYNK-Exo by LN-229. (Left) Viability of LN-229 after direct siRNA transfection with negative control or TGF-β siRNA, or with treatment of negative or TGF-β siCYNK-Exo. (Right) Percent cells positive for tagged-siRNA after direct transfection with negative control or TGF-β siRNA, or with treatment of negative or TGF-β siCYNK-Exo.

FIG. 11. Silencing TGF-β in U251 by siCYNK-Exo. (Left) Expression of TGF-β as determined by RT-qPCR in U251 after direct siRNA transfection with negative control or TGF-β siRNA, or with treatment of negative or TGF-β siCYNK-Exo. (Right) Concentration of TGF-β secreted by U251 after direct siRNA transfection with negative control or TGF-β siRNA, or with treatment of negative or TGF-β siCYNK-Exo. FIG. 12. Silencing TGF-β in LN-229 by siCYNK-Exo. (Left) Expression of TGF-β as determined by RT-qPCR in LN-229 after direct siRNA transfection with negative control or TGF-β siRNA, or with treatment of negative or TGF-β SiCYNK-Exo. (Right) Concentration of TGF-β secreted by LN-229 after direct siRNA transfection with negative control or TGF-β siRNA, or with treatment of negative or TGF-β siCYNK-Exo.

FIG. 13. CYNK-001 Cytotoxicity is Improved Following siCYNK-Exo Treatment. (Left) Cytotoxicity of U251 transfected with siRNA for 24 hours followed by addition of CYNK-001 at an E:T of 0.5:1. (Right) Cytotoxicity of U251 treated with siCYNK-Exo followed by addition of CYNK-001 at an E:T of 0.5:1.

FIG. 14. siCYNK-Exo Increases Cytokine Production When Combined with CYNK-001. Concentration of (A) TNFα, (B) GM-CSF, (C) INFγ, (D) perforin, (E) granzyme A and (F) granzyme B secreted from treated U251 alone or when treated followed by CYNK-001 addition.

FIG. 15. TGF-β siCYNK-Exo Increases CYNK-001 Cytokine Production. Changes in secretion of (A) perforin, (B) granzyme A and (C) granzyme B when CYNK-001 is added to U251 treated with negative control siCYNK-Exo versus TGF-β siCYNK-Exo.

### DETAILED DESCRIPTION

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure.

All numerical designations, e.g., volume, mass, number of resin particles, etc. are approximations which are varied by (+) or (−) by increments of 1.0 or 0.1, as appropriate. It is to be understood, although not always explicitly stated, that all numerical designations are preceded by the term “about”.

Numerous terms and phrases are used throughout the instant specification and claims and are defined below.

“About” and “approximately” are interchangeable and mean plus or minus a percent (e.g., ±5%) of the number, parameter, or characteristic so qualified, which would be understood as appropriate by a skilled artisan to the scientific context in which the term is utilized.

As used here, the singular form “a”, “an” and “the” include plural reference unless the context clearly dictates otherwise.

As used herein, the terms “comprising”, “comprises” and “comprise” are intended to mean that the compositions, preparations, and methods disclosed herein include recited elements, but do not exclude others.

As used herein, “administration” or “administrating” means providing an exosomal composition and a placental derived CYNK-001 cell systemically or locally to cancer cells.

As used herein, the term “down-regulate” refers to a process in which a cell decreases the production and quantities of its cellular components, such as RNA and proteins, in response to an external stimulus.

As used herein the phrase “effective amount” refers to an amount of a therapeutic composition (e.g., an exosomal composition of the instant disclosure and a pharmaceutically vehicle, carrier, or excipient, as well as CYNK-001 cells) sufficient to produce a measurable biological response. Actual dosage levels of active ingredients in a therapeutic composition of the present disclosure can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, N.J.; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

As used herein, the phrase “chemotherapeutic agent” refers to agents that directly or indirectly inhibit the uncontrolled growth and proliferation of cancer cells. They are classified according to their mechanism of action. Examples of such classifications include alkylating agents, antineoplastic agents, topoisomerase inhibitors, taxanes, hormone analogs, antimetabolites, plant alkaloids, epipodophyllotoxins, anti-tumor antibiotics, anthracyclines, and biologics As used herein, the phrase “small molecule” refers to compounds ranging from 0.1 to 1.0 kDA, which are created through chemical synthesis or derived from natural products produced by bacteria, fungi, and plants.

As used herein, the term “biologic” includes a wide range of products such as vaccines, blood and blood components, allergenics, somatic cells, gene therapy, tissues, and recombinant therapeutic proteins. Biologics can be composed of sugars, proteins, nucleic acids or complex combinations of these substances, or may be living entities such as cells and tissues. Biologics are isolated from a variety of natural sourcesp13 human, animal, or microorganism—and may be produced by biotechnology methods and other technologies. Gene-based and cellular biologics, for example, often are at the forefront of biomedical research, and may be used to treat a variety of medical conditions for which no other treatments are available.

As used herein, the term “exosome” refers to naturally existing nanoparticles that are secreted endogenously by many types of in vitro cell cultures and in vivo cells, and are commonly found in vivo in body fluids, such as blood, urine and malignant ascites. Exosomes are cup-like multivesicular bodies (MVBs) varying in size between 30-100 nm (12). MVBs are specialized endosomes in the endocytosis pathway of cells and are formed by inward budding and scission of vesicles from the limiting membranes into the endosomal lumen (13). During the formation of MVBs, transmembrane and peripheral membrane proteins are absorbed into the vesicle membrane, and at the same time, cytosolic components are also embedded in the vesicles. As this process progresses, the MVBs ultimately fuse with the cellular membrane, triggering the release of the exosomes from the cells.

During this process, unwanted molecules are eliminated from cells. However, cytosolic and plasma membrane proteins are also incorporated during this process into the exosomes, resulting in exosomes having particle size properties, lipid bilayer functional properties, and other unique functional properties that allow the exosomes to potentially function as effective nanoparticle carriers of therapeutic agents. In this regard, it has now been discovered that exosomes can be used as part of a specific nanoparticle-therapeutic agent delivery system that is able to deliver a therapeutic agent to target cells and tissues, while also retaining the biological activity of the therapeutic agents. In particular, it has been observed that the formation of exosome-therapeutic agent complexes results in an increase in the solubility and stability of the therapeutic agents as well as an increase in their bioavailability, all of which have been major obstacles in the treatment of inflammatory disorders and cancers.

As explained herein, exosomes of an exosomal composition of the instant composition comprise an siRNA nucleotide sequence that targets the expression of TGF-β. In a particular embodiment, the nucleotide sequence is SEQ ID NO: 1, and the nucleotide sequence is encapsulated by the exosome. The phrase “encapsulated by an exosome,” or grammatical variations thereof is used interchangeably herein with the phrase exosomal composition” refers to exosomes whose lipid bilayer surrounds an siRNA nucleotide sequence targeting the expression of TGF-β. For example, a reference to “exosomal curcumin” refers to an exosome whose lipid bilayer encapsulates or surrounds an effective amount of curcumin.

In some embodiments, the encapsulation of an siRNA nucleotide sequence that targets the expression of TGF-β within exosomes can be achieved by first mixing such an siRNA with isolated exosomes secreted from a placental derived natural killer (CYNK) cell, e.g., CYNK-001, in a suitable buffered solution, such as phosphate-buffered saline (PBS). After a period of incubation sufficient to allow the therapeutic agent to become encapsulated during the incubation period, the exosome/therapeutic agent mixture is then subjected to a sucrose gradient (e.g., and 8, 30, 45, and 60% sucrose gradient) to separate the free therapeutic agent from the therapeutic agents encapsulated within the exosomes, and a centrifugation step to isolate the exosomes. After this centrifugation step, the exosomes that encapsulate the siRNA are seen as a band in the sucrose gradient such that they can then be collected, washed, and dissolved in a suitable solution for use as described herein below. A particular example of constructing exosomes of an exosomal composition of the instant disclosure is described in the Example.

As used herein, CYNK-001 cells are cells that are disclosed and described in US published patent application US20220265712, which is hereby incorporated by reference in its entirety.

The present disclosure may be better understood by reference to the following non-limiting examples, which are provided as exemplary of the disclosure. The following examples are presented in order to more fully illustrate the preferred embodiments of the disclosure. They should in no way be construed, however, as limiting the broad scope of the disclosure.

### EXAMPLE

### Isolating and Modifying Exosomes from Human Placental CD34+ Derived Natural Killer (CYNK-001) for Anti-Tumor Treatment

### Introduction

Celularity Inc. has developed a novel platform that enables production of an allogenic, placental derived NK cells for the treatment of various hematological and solid tumor malignancies. NK-derived exosomes have gained notability for their anti-tumor activity against leukemia, melanoma and GBM [1-3]. Exosomes are nano-scaled extracellular vesicles that are natural carriers of RNA and protein [4]. Engineering exosomes to carry short interfering RNA (siRNA) means they can have binary effect against tumor cells [5].

Here we provide evidence of successfully isolated CYNK-001-derived exosomes (CYNK-Exo) as defined by size and expression profile. CYNK-001 cells are disclosed and described in US Published Patent Application US20220265712, which is hereby incorporated by reference in its entirety. Here, we provide data to support that CYNK-Exo have inherent anti-tumor effects against solid tumors, specifically gastric cancer, and glioblastoma (GBM). Further, loading CYNK-Exo with TGF-β SiRNA (siCYNK-Exo) can effectively knockdown expression in tumor cells and thereby reduce tumor-immunosuppression thus enhancing CYNK-001 activity.

### Materials and Methods

Isolation of CYNK-Exo from Conditioned Media

The Invitrogen Total Isolation Reagent was used to isolate exosomes using reagent-based isolation. Briefly, conditioned media was centrifuged for 30 minutes at 3,000×g to remove dead cells, followed by overnight incubation with Isolation Reagent at 4° C. and then 1 hour spin at 10,000 g to pellet CYNK-Exo. To isolate exosomes by ultracentrifugation, conditioned media was centrifuged for 30 minutes at 3,000×g to remove dead cells, followed by 1 hour at 10,000×g to remove cellular debris, and lastly, for 2 hours at 100,000×g to pellet CYNK-Exo. To isolate exosomes by sucrose gradient isolation, conditioned media was filtered through a 0.2 um filter. Thereafter, 30 mL of filtered media was layered atop 4 mL of 30% sucrose without mixing the two layers and ultracentrifuged for 90 minutes at 100,000×g at 4° C. The bottom sucrose layer was collected and washed with PBS followed by pelleting the CYNK-Exo by ultracentrifugation for 90 minutes at 100,00×g at 4° C. To isolate exosomes by size exclusion chromatography (SEC), the automated IZON column was used. Briefly, columns were equilibrated with PBS followed by 10 mL of ultracentrifuged CYNK-Exo. To test which fraction was the sample of interest, 1 mL fractions were collected and tested. Data below uses pooled fractions 6-8. All CYNK-Exo pellets were resuspended in PBS, aliquoted and stored in −80° C.

### Characterization of CYNK-Exo

To characterize CYNK-Exo, isolated exosomes were captured to beads using tetraspanin biotinylated antibodies according to manufacturer's protocol (System Biosciences, catalog #EXOFLOW100A-1). Briefly, CYNK-Exo incubated with antibodies overnight to ensure capture. The following day CYNK-Exo were stained for CD81 (Biolegend, catalog #349511), HLA-I (R&D Systems, catalog #FAB7098A), HLA-II (Biolegend, catalog #343312), CD11a (BD, catalog #561387), CD16 (BD, catalog #555407), CD47 (Biolegend, catalog #323123), CD56(BD, catalog #557919), CD226(BD, catalog #564796) and FasL (Biolegend, catalog #306411). CYNK-Exo bound to beads were washed and then labelled with FITC to indicate positive binding of exosomes to beads.

CYNK-Exo concentration was measured by microBCA kit purchased from Invitrogen (catalog #23235). To measure particle size and concentration, samples were diluted 1:100 in PBS and measured by ZetaView using on light scatter technology.

To characterize cytokines, supernatant from cultures or approximately 25 ug of exosomes were analyzed by Luminex xMAP technology. Briefly, 200 uL of Assay buffer is added per well and incubated on the shaker for 10 minutes at room temperature. Assay buffer is decanted, and residue is removed by inverting the plate and tapping smartly onto absorbent towels. 25 uL of sample, Assay buffer and Bead Mix is added to each well to make the final volume 75 uL per well. Plates are sealed, wrapped with foil, and placed in 4° C. to incubate in the dark overnight. The next day, plates are washed two times and 25 uL of Detection Antibody added per well. After 1 hour incubation at room temperature, 25 uL of Streptavidin-Phycoerythrin is added per well and incubate for an additional 30 minutes at room temperature. The plate is washed two more times and beads are resuspended in 150 uL of Sheath Fluid for acquisition using the FLEXMAP 3D.

### Cytotoxic Assays

The xCelligence Real-Time Cell Analysis (RTCA) system was used to measure cytotoxicity based on cellular impedance readout as Cell Index (CI) to monitor real-time changes in cell number. Target cells were plated in 96-well E-Plates for 24 hours. Thereafter, CYNK-Exo were added based on protein concentration and target cell impedance was monitored for an additional 72 hours. Data was normalized to the untreated target cells and extrapolated as % cytotoxicity. For experiments using CYNK-001, cells were thawed and recovered in the incubator for three days before adding to target cells.

The LDH Assay Kit (Cytotoxicity) (Abcam, catalog #ab65393) was used to assess the cytotoxic effects of test compounds on cultured cells. The assay kit is based on the measurement of lactate dehydrogenase (LDH) released from damaged cells into the culture medium. The method involves seeding cells in a 96-well plate, treating them with various doses of CYNK-Exo, and incubating for the indicated time. The supernatant was collected and the amount of LDH activity was measured using a microplate reader. The amount of LDH activity in the supernatant is directly proportional to the number of damaged cells.

The final method of assessing cytotoxicity on target cells by CYNK-Exo was 7AAD and annexin V staining, a commonly used method for the detection of viable, apoptotic, necrotic and/or dead cells.

### Transfecting Cynk-exo With Sirna

The Exo-Fect Exosome Transfection Kit from System Biosciences (catalog #EXFT200A-1) was used to deliver nucleic acids into CYNK-Exo. The kit includes all necessary reagents for the transfection process, including Exo-Fect reagent and transfection buffer. The protocol involves incubating exosomes with the nucleic acid of interest, which in the instant matter was siRNA for TGF-β having the nucleotide sequence of GGCUCAAGUUAAAAGUGGATT (SEQ ID NO: 1) labeled with Cyanine3 (Cy3) at the N-terminus guanine, and Exo-Fect reagent, followed by incubation with the provided transfection buffer. After the transfection, exosomes are harvested, and any residual RNA is washed out. As positive and negative controls, cells were directly transfected with siRNA using RNAi Max Lipofectamine (Invitrogen, catalog #13778100).

siCYNK-Exo Uptake Assay

Tumor cells seeded in 96-well plates were treated with 25 μg of siCYNK-Exo or equal volumes of PBS for 24, 48 and 72 hours. Thereafter, cells were washed with PBS and detached using trypsin. Cells were stained with 7AAD, and data acquired by BD FACSCanto10. Cells positive in the PE channel indicated siRNA uptake.

### Cytokine Analysis by Enzyme-Linked Immunosorbent Assay (ELISA)

The Human/Mouse/Rat/Porcine/Canine TGF-β eta 1 Quantikine ELISA from R&D Systems (catalog #DB100C) was used to quantitatively measure TGF-β eta 1 levels in biological samples. The kit includes pre-coated microplates, a standard curve, and all necessary reagents to perform the assay. The assay was performed according to the manufacturer's instructions. In brief, the samples and standards were added to the wells and incubated with a biotinylated antibody specific for TGF-β eta 1. After washing, streptavidin-HRP conjugate was added to the wells and incubated to bind the biotinylated antibody. Following a final washing step, substrate solution was added to the wells to induce a colorimetric reaction. The reaction was stopped by addition of stop solution and the absorbance was measured at 450 nm. The amount of TGF-β eta 1 in each sample was determined by comparing the absorbance values to the standard curve.

### Results

### 1. Characterization of CYNK-Exo

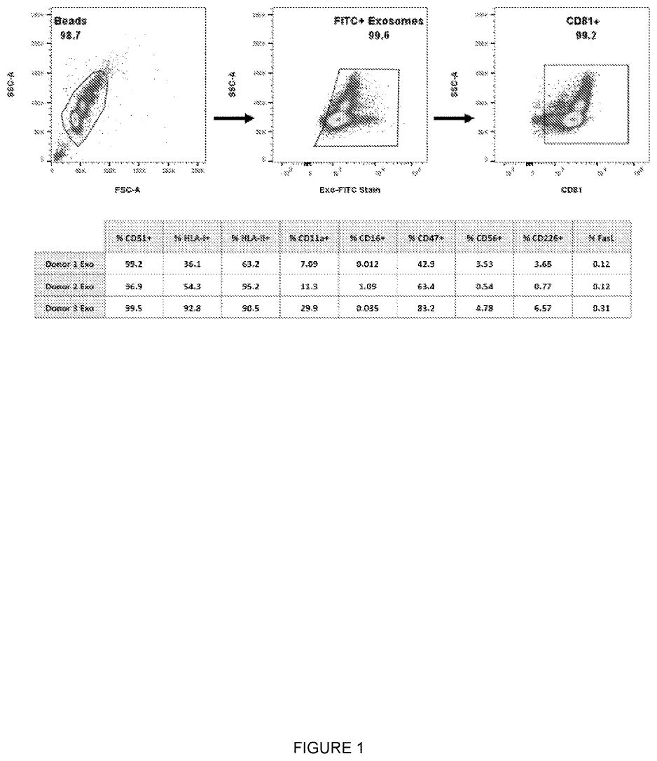

Exosomes are distinct from other extracellular vesicles by their size and surface markers. CYNK-Exo were isolated from CYNK-001 conditioned media and assessed for the presence of exosome-and NK-specific markers by flow cytometry (FIG. 1). Table 1, located below the gating strategy, summarizes the expression levels of various markers on CYNK-Exo. The data show that CYNK-Exo are nearly 100% positive for CD81, an exosome specific marker, 61.1% positive for HLA-1, 83.0% positive for HLA-II, 16.1% positive for CD11a, and 63.2% positive for CD 47. However, less than 5% of CYNK-Exo donors expressed CD16, CD56, CD226, and FASL (n=3 CYNK-Exo donors).

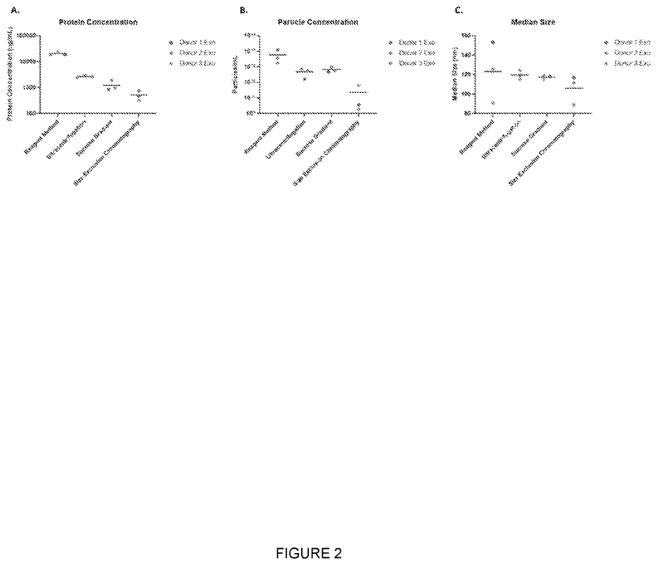

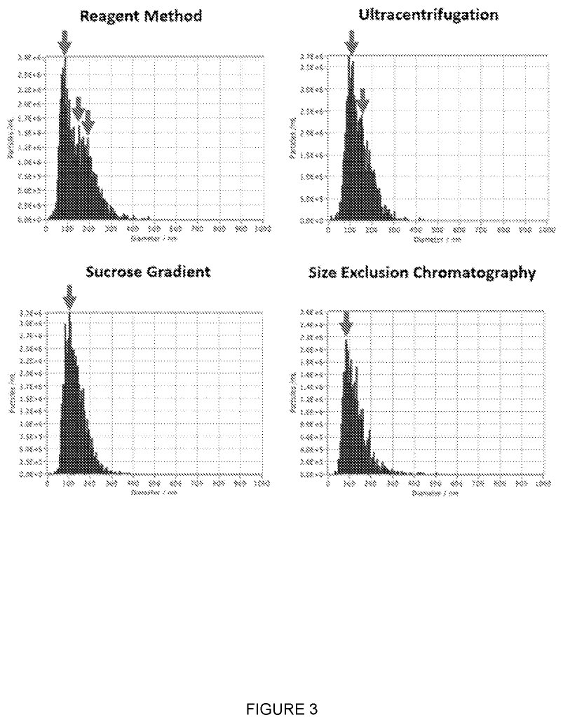

CYNK-Exo isolated by different methods were compared using microBCA to assess protein concentration and nanoparticle tracking to assess size distribution and concentration. The reagent-based method yielded the highest protein concentration (ug/mL) of CYNK-Exo, whereas SEC resulted in the lowest protein concentration (FIG. 2A). The concentration of particles/mL and the median size of CYNK-Exo were determined by ZetaView, revealing that the reagent-based method had the highest concentration of particles/mL, whereas SEC had the lowest (FIG. 2B). The reagent-based method showed a large variation in median size of CYNK-Exo, which was not observed with the sucrose gradient method that had the most consistent median particle size across the three donors (FIG. 2C). An exemplary size distribution of vesicles for each isolation method illustrates the homogeneity of CYNK-Exo isolated by sucrose gradient and SEC (FIG. 3). The reagent-based and ultracentrifugation methods displayed several peaks, indicating a range of microvesicles that may not necessarily be exosomes.

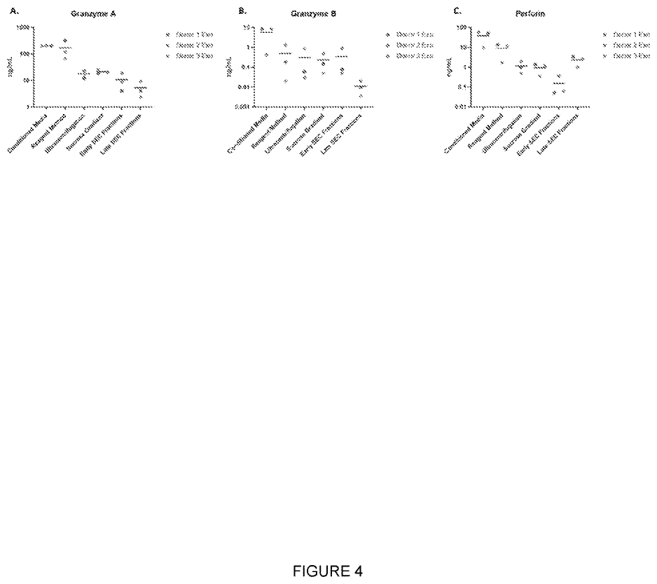

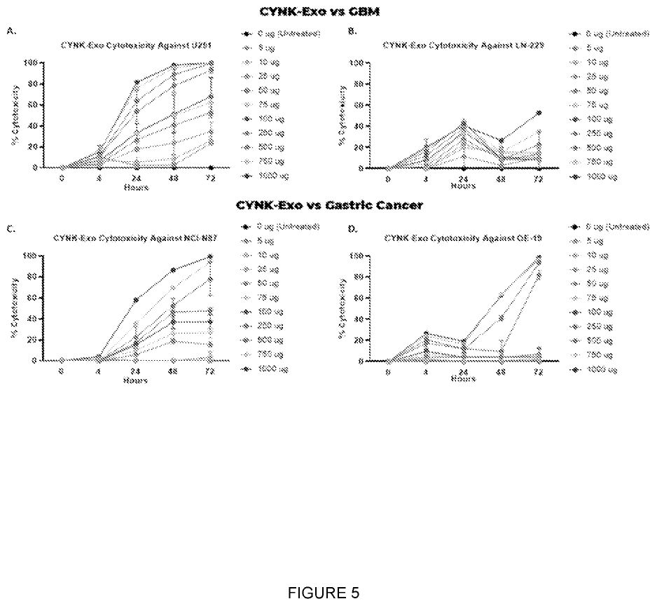

Next, we examined the levels of granzyme A, granzyme B, and perforin in the conditioned media of CYNK-001 and in CYNK-Exo derived from different isolation methods (FIG. 4). Our findings indicate that the composition of CYNK-Exo obtained through ultracentrifugation and sucrose-gradient isolation methods are comparable in terms of these cytotoxicity-associated molecules. However, sucrose-gradient isolation results in more consistent exosome size and exhibits greater cytotoxicity than ultracentrifugation isolation (FIG. 2). Taken together, future studies utilized sucrose gradient isolation to collect and evaluate CYNK-Exo.

### 2. CYNK-Exo Possess Cytotoxic Activity

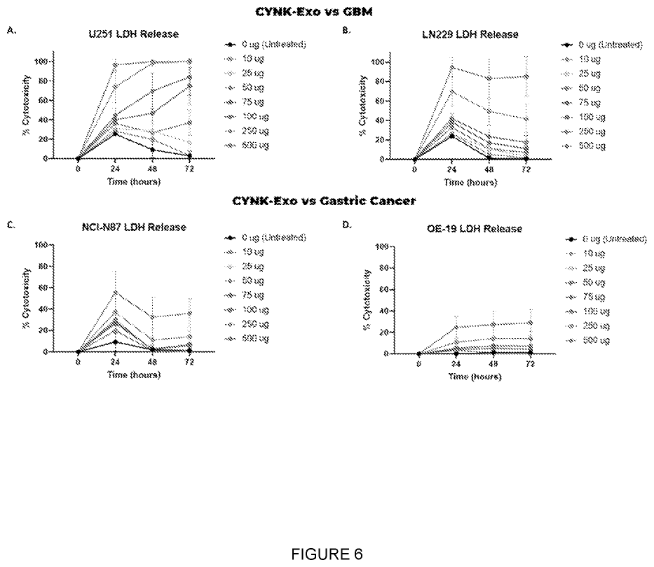

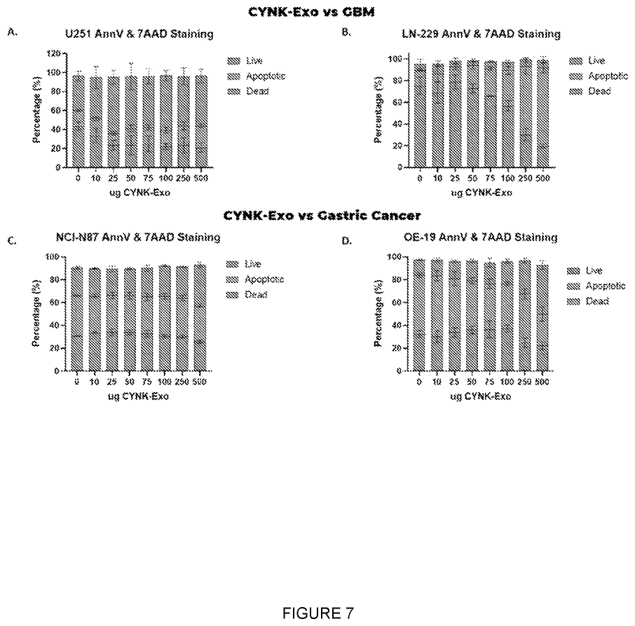

The following data investigated the cytotoxic effects of CYNK-Exo on various cancer cell lines. The real-time cell analysis system xCelligence was employed to monitor the effects of CYNK-Exo on tumor cells, as illustrated in FIG. 5. The results demonstrate that CYNK-Exo induce cytotoxicity on gastric cancer cells with as little as 50μg inducing 15% cytotoxicity in NCI-N87 cells, and 250 μg inducing 80% cytotoxicity in OE-19 cells after 72 hours of exposure. Furthermore, CYNK-Exo effectively induced cell death in two GBM cell lines, U251 and LN-229, as revealed by the LDH release assay, as shown in FIG. 6. Similar to xCelligence, LN-229 and OE-19 cells exhibited a higher degree of resistance to the cytotoxic effects of CYNK-Exo compared to U251 and NCI-N87 cells. To further assess the cellular response to CYNK-Exo treatment, 7AAD and annexin V staining was performed to determine the percentage of live, apoptotic and dead cells, as depicted in FIG. 7. Staining of the U251 cells shows an increase in the dead population in a dose-dependent manner. LN-229 had decreased live and increased apoptotic cells as it exceeded 75 ug of CYNK-Exo. There were marginal differences in the gastric cell lines, NCI-N87 and OE-19, which suggest that cells should be evaluated longer than 24 hours. Taken together, the data suggest that CYNK-Exo holds great potential as a cytotoxic therapeutic agent against various types of cancer cells.

3. Loading CYNK-Exo with siRNA

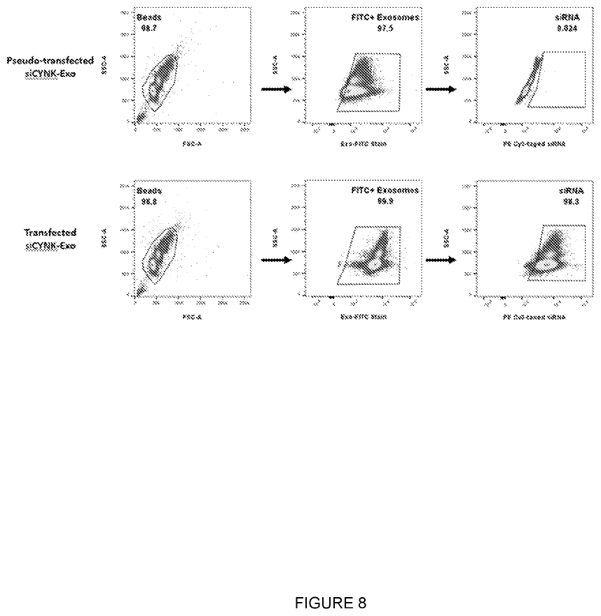

In this study, we aimed to develop dual-purposed exosomes that can simultaneously deliver therapeutic siRNA molecules and exert cytotoxic effects on cancer cells. For this purpose, we loaded CYNK-Exo with Cy3-tagged siRNA using the Exo-Fect Exosome Transfection Kit. The efficiency of siRNA loading was evaluated by analyzing the fluorescence signal intensity in the PE channel using flow cytometry, as shown in FIG. 8. To ensure that the detected signal was specific to exosome-encapsulated siRNA, we performed a control experiment using pseudo-transfected siCYNK-Exo, which consisted of CYNK-Exo and siRNA without the transfection reagent. The results confirmed that residual siRNA was effectively removed from the reaction and not loaded inside the exosomes. In contrast, CYNK-Exo incubated with siRNA and transfection reagent exhibited a high degree of siRNA loading efficiency, as evidenced by the positive fluorescence signal in the PE channel. These findings suggest that siRNA can be effectively delivered into CYNK-Exo using the Exo-Fect Exosome Transfection Kit, thus creating a promising platform for developing novel exosome-based therapeutic strategies.

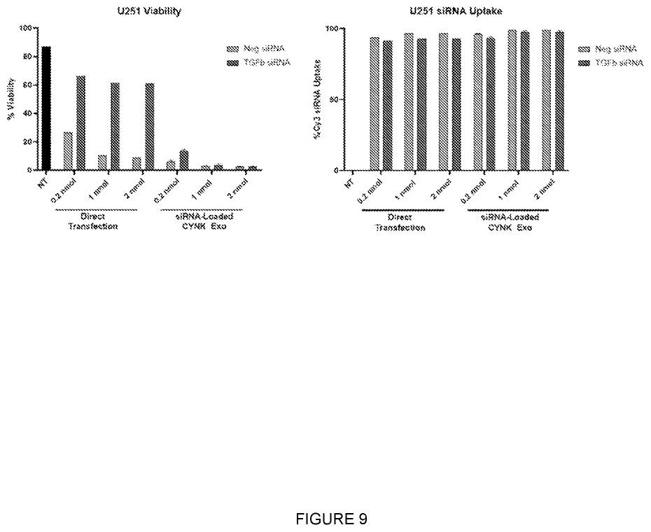

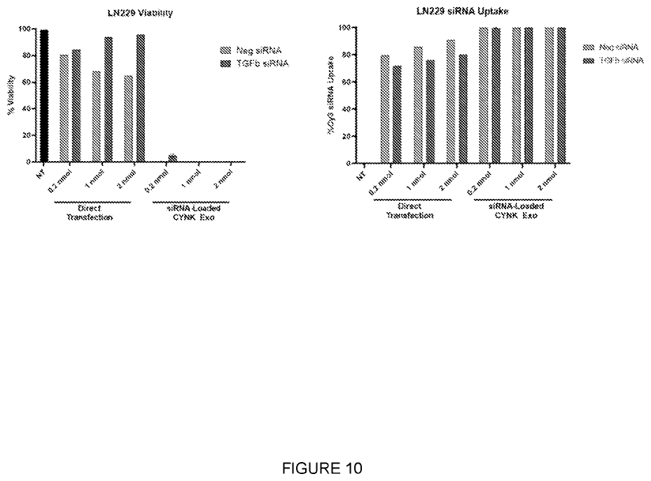

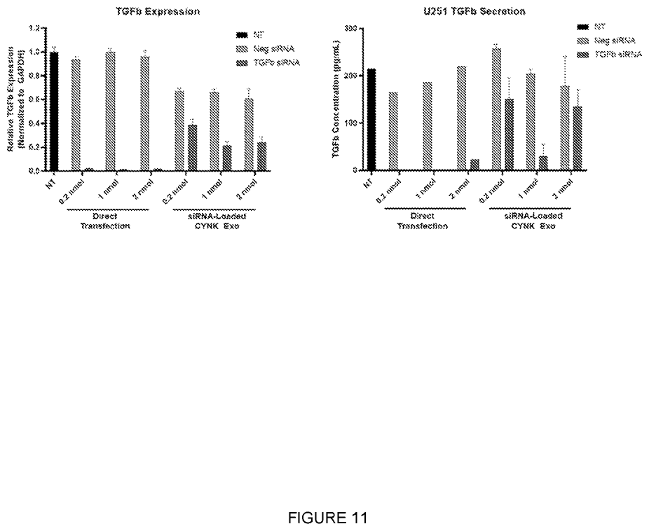

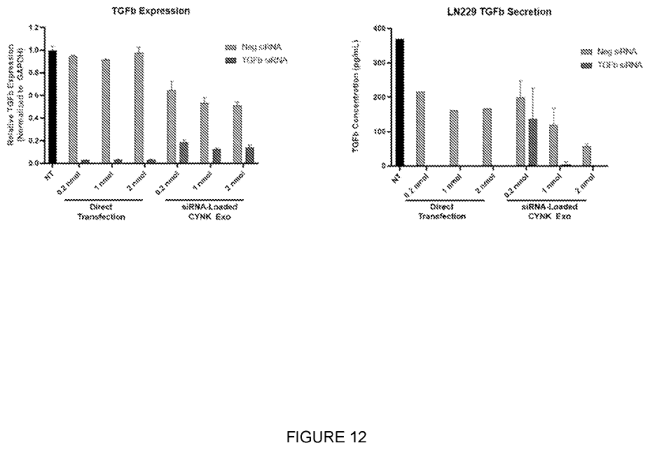

To optimize the knockdown efficiency of TGF-β using siRNA-loaded CYNK-Exo, we performed a series of experiments to determine the optimal siRNA concentration for efficient delivery and maximal therapeutic efficacy. The results of these experiments are presented in FIGS. 9-10. Specifically, we tested various concentrations of TGF-β siRNA-loaded CYNK-Exo in GBM cell lines U251 and LN-229 and assessed their cytotoxic effects on cell viability using a standard cell viability assay. As expected, we observed that cells treated with either negative control or TGF-β siRNA-loaded CYNK-Exo had significantly reduced cell viability, consistent with the inherent cytotoxicity of CYNK-Exo. Notably, we confirmed the uptake of exosomes by cells transfected directly with siRNA or treated with siRNA-loaded CYNK-Exo, as evidenced by the strong Cy3-tagged siRNA signal observed in both cases. Furthermore, we observed a decrease in cell viability with increasing concentrations of TGF-β siRNA-loaded CYNK-Exo, indicating that the siRNA was effectively delivered into the cells and exerted its cytotoxic effect. Overall, these findings suggest that optimizing the siRNA concentration in CYNK-Exo can significantly enhance their therapeutic potential for targeting TGF-β in GBM.

4. Uptake of siCYK-Exo by Tumor Cells

U251 cells treated with siCYNK-Exo showed efficient uptake with 97.7% Cy 3+ cells. RT-qPCR results demonstrated 78.6% reduction in TGF-β mRNA expression and ELISA results demonstrated 71.8% reduction in TGF-β secretion. Similar findings were demonstrated on LN-229 GBM cell line.

5. Silencing TGF-β in GBM by siCYNK-Exo

To evaluate the efficiency of TGF-β siCYNK-Exo on TGF-β, the RNA expression and TGF-β secretion were analyzed using RT-qPCR and ELISA, respectively. To this end, a non-transfected control and direct cellular transfection controls were employed. The results of both RT-qPCR and ELISA analyses revealed that siCYNK-Exo could reduce TGF-β expression with a minimum of 0.2 nmol/ug CYNK-Exo. Future investigations will utilize 1 nmol of siRNA to load CYNK-Exo, while testing lower doses of CYNK-Exo to determine the minimum threshold for its activity.

6. CYNK-001 Cytotoxicity is Improved Following siCYNK-Exo Treatment

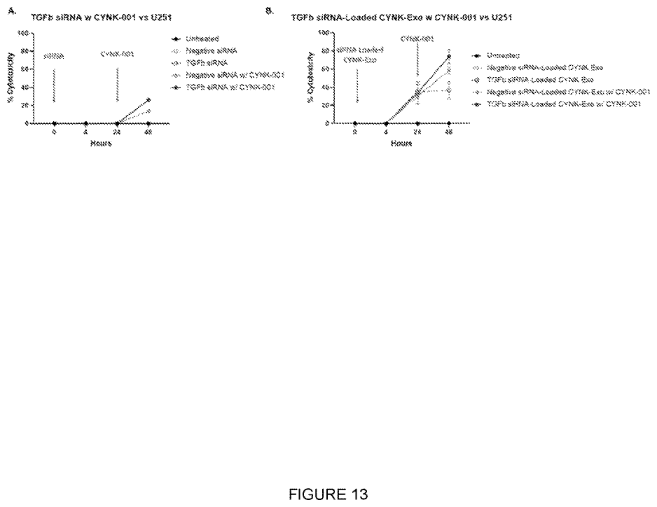

U251 tumor cells were subjected to a study aimed at investigating the potential of decreasing TGF-β expression to enhance the cytotoxicity of CYNK-001 (FIG. 13). The cells were treated with either siRNA or siCYNK-Exo for 24 hours, followed by the addition of CYNK-001 at an E:T ratio of 0.5:1. FIG. 13A provides compelling evidence that knocking down TGF-β expression can enhance the cytotoxicity of CYNK-001. Furthermore, FIG. 13B demonstrates that while the addition of CYNK-Exo alone exerts an inherent cytotoxic effect against U251, the inclusion of TGF-β siCYNK-Exo leads to a marked improvement in the efficacy of CYNK-001-mediated killing compared to negative siCYNK-Exo. Collectively, these findings suggest that TGF-β siCYNK-Exo exhibits a synergistic cytotoxicity with CYNK-001, providing impetus for further studies aimed at exploring the potential of TGF-β siCYNK-Exo as a therapeutic strategy for cancer.

7 Silencing TGF-β in GBM by siCYNK-Exo increases CYNK-001 Cytokine Production

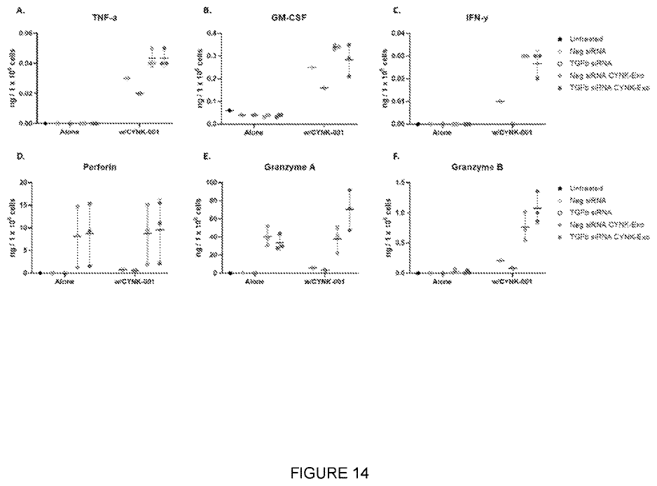

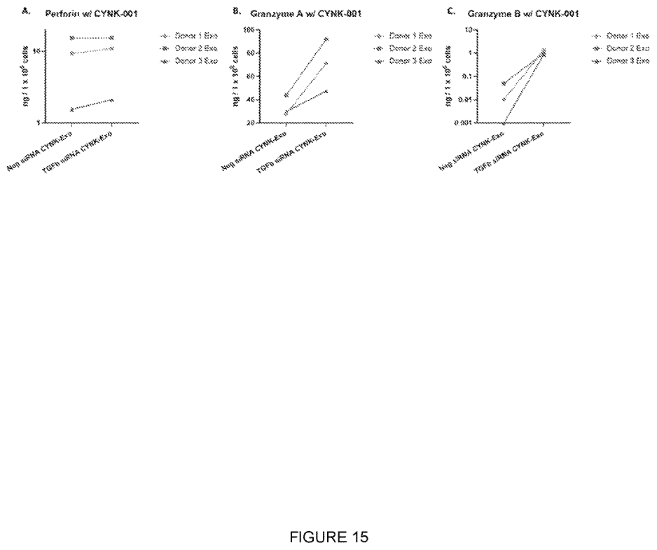

To investigate whether the increased cytotoxicity observed with the combination of CYNK-001 and TGF-β siCYNK-Exo was due to cytokine production, cytokine secretion was analyzed (FIGS. 14-15). Our data reveals that the treatment of U251 cells with either negative control siRNA-loaded CYNK-Exo or TGF-β siCYNK-Exo improves the secretion of inflammatory cytokines from CYNK-001, suggesting that the use of CYNK-Exo in combination with CYNK-001 may exhibit a synergistic effect even without targeting TGF-β with siRNA. Additionally, FIG. 15 shows that perforin, granzyme A, and granzyme B levels are increased when U251 cells are first treated with TGF-β siCYNK-Exo. These findings support our hypothesis that TGF-β siCYNK-Exo can synergize with CYNK-001 to improve cancer cell death. Further investigations will aim to optimize the transfection of CYNK-Exo with siRNA to enhance knockdown efficiency.

### Conclusion

Data presented in this study suggests that CYNK-Exo holds great potential as a cytotoxic therapeutic agent against various types of cancer cells. CYNK-Exo were shown to be distinct from other extracellular vesicles by their size and surface markers, and their isolation methods were compared in terms of protein concentration, size distribution, and cytotoxicity-associated molecules. Sucrose gradient isolation was found to be the most consistent method for isolating CYNK-Exo and exhibited increased cytotoxicity. CYNK-Exo induced cytotoxicity on gastric cancer and GBM cell lines in a dose-and time-dependent manner. TGF-β siCYNK-Exo and were able to deliver therapeutic siRNA to tumor cells and knockdown cytokine production. Using TGF-β siCYNK-Exo with CYNK-001 had a synergistic effect in killing tumor cells. These findings suggest that CYNK-Exo could be used as a novel exosome-based therapeutic strategy for cancer treatment.

### References

1. Samara, A., Anbar, M., Shapira, S., Zemlyansky, A., Zozovsky, A., Raanani, P., Granot, G., Rozovski, U. Using natural killer cell-derived exosomes as a cell-free therapy for leukemia. Hematol Oncol., doi: 10.1002/hon.3111, 2022. PMID: 26451254.
2. Zhu, L., Kalimuthu, S., Oh, J. M., Gangadaran, P., Baek, S. H., Jeong, S. Y., Lee, S. W., Lee, J., Ahn, B. C. Enhancement of antitumor potency of extracellular vesicles derived from natural killer cells by IL-15 priming. Biomaterials, 190-191:38-50., 2019. PMID: 30391801.
3. Zhu, L., Kalimuthu, S., Gangadaran, P., Oh, J. M., Lee, H. W., Baek, S. H., Jeong, S. Y., Lee, S-W., Lee, J., Ahn, B. C. Exosomes derived from natural killer cells exert therapeutic effect in melanoma. Theranostics, 7(10), 2732-2745, 2017. PMID: 28819459.
4. LeBleu, V. S., Kalluri, R. The biology, function, and biomedical applications of exosomes. Science, 7; 367(6478), 2020. PMID: 32029601.
5. Slattery K., Woods E., Zaiatz-Bittencourt V., Marks, S., Chew, S., Conroy, M., Goggin, C., MacEochagain., Kennedy, J., Lucas, Sophie., Finlay, D. K., Gardiner, C. M. TGF-β drives NK cell metabolic dysfunction in human metastatic breast cancer. Journal for ImmunoTherapy of Cancer, 9: e002044, 2021. PMID: 33568351.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

1. An exosomal composition comprising exosomes that comprise the nucleotide sequence of GGCUCAAGUUAAAAGUGGATT (SEQ ID NO: 1) and a pharmaceutically acceptable excipient, wherein said exosomes are secreted from placental derived natural killer (CYNK) cells.

2. The exosomal composition of claim 1, wherein the CYNK cells are placental CD34+ cell-derived natural killer (NK) cells.

3. The exosomal composition of claim 1 wherein the CYNK cells are CYNK-001 cells.

4. A method of down-regulating expression of TGF-β in cancer cells in a subject, comprising administering an effective amount of the exosomal composition of claim 1.

5. A method for treating cancer, comprising the steps of: (i) administering to a subject in need thereof an effective amount of an exosomal composition comprising: (a) exosomes secreted from placental derived natural killer (CYNK) cells, wherein the exosomes comprise the nucleotide sequence of GGCUCAAGUUAAAAGUGGATT (SEQ ID NO: 1); and (b) a pharmaceutically acceptable excipient; and (ii) administering to the subject effective amount of a population of the placental derived natural killer (CYNK) cells.

6. A method of treating cancer, comprising administering a therapeutically effective amount of an exosomal composition that comprises: (a) exosomes secreted from placental derived natural killer (CYNK) cells, wherein the exosomes comprise the nucleotide sequence of GGCUCAAGUUAAAAGUGGATT (SEQ ID NO: 1); and (b) a pharmaceutically acceptable excipient.

7. The method of claim 5, wherein the cancer is selected from the group consisting of skin cancer, gastric cancer, head and neck cancer, colon cancer, breast cancer, brain cancer, and lung cancer.

8. The method of claim 7, wherein the cancer is a brain cancer.

9. The method of claim 8, wherein the brain cancer is glioblastoma (GBM).

10. The method of claim 5, wherein the placental derived natural killer (CYNK) cells are placental CD34+ cell-derived natural killer (NK) cells.

11. The method of claim 10, wherein the placental CD34+ cell-derived natural killer (NK) cells are CYNK-001 cells.

12. The method of claim 5, further comprising the step of administering an effective amount of a chemotherapeutic agent.

13. The method of claim 12, wherein the chemotherapeutic is a small molecule, a biologic, or a combination thereof.

14. The method of claim 6, wherein the cancer is selected from the group consisting of skin cancer, gastric cancer, head and neck cancer, colon cancer, breast cancer, brain cancer, and lung cancer.

15. The method of claim 14, wherein the cancer is a brain cancer.

16. The method of claim 15, wherein the brain cancer is glioblastoma (GBM).

17. The method of claim 6, wherein the placental derived natural killer (CYNK) cells are placental CD34+ cell-derived natural killer (NK) cells.

18. The method of claim 17, wherein the placental CD34+ cell-derived natural killer (NK) cells are CYNK-001 cells.

19. The method of claim 6, further comprising the step of administering an effective amount of a chemotherapeutic agent.

20. The method of claim 19, wherein the chemotherapeutic is a small molecule, a biologic, or a combination thereof.
